(19) United States
(12) Patent Application Publication
(10) Pub. No.: US 2026/0272818 A1
(43) Pub. Date: Sep. 17, 2026

(54) HAEMATOCOCCUS EXTRACT SKIN CARE COMPOSITION

(71) Applicant: ALGAKTIV, S.L., Barcelona (ES)

(72) Inventors: Noemí GARCÍA-DELGADO BANCHS, Esplugues del Llobregat, Barcelona (ES); Jaume MERCADÉ ROCA, Barcelona (ES); Eugènia RUIZ CÁNOVAS, El Prat de Llobregat, Barcelona (ES); Gerard SALA I MAYENCH, El Prat de Llobregat, Barcelona (ES); Jordi AYATS FORRELLAT, Terrassa, Barcelona (ES)

(21) Appl. No.: 19/469,034

(22) PCT Filed: Mar. 27, 2024
(86) PCT No.: PCT/EP2024/058249

§ 371 Date: Sep. 25, 2025

(30) Foreign Application Priority Data
Mar. 27, 2023 (EP) 23382283.2

(51) Int. Cl. A61K 8/9722; A61K 8/73; A61Q 19/08

(52) U.S. Cl. CPC A61K 8/9722; A61K 8/735; A61Q 19/08

(57) ABSTRACT

A combination comprising Haematococcus pluvialis microalgae extract containing polyamines such as spermidine and collagen amino acids and the hyaluronic acid. The invention relates to the field of cosmetics products and uses thereof in cosmetic methods as well as in the preparation of, cosmeceutical or pharmaceutical composition.

### FIELD OF THE INVENTION

The invention relates to the field of natural products and uses thereof in cosmetic methods as well as in the preparation of, cosmeceutical or pharmaceutical composition.

### BACKGROUND ART

The concern to preserve for as long as possible a young-looking skin is a preoccupation of the majority of women and is also increasingly affecting the men. In order to meet this expectation, cosmetic compositions aimed at preventing and/or treating the signs of skin ageing have therefore been developed.

Skin aging is defined by all the alterations of the cutaneous covering resulting from the accumulation over the years of the gradual modifications of its various constituents.

It results, inter alia, in a flattening of the epidermis and of the dermo-epidermal junction and in the dermis, though, on the one hand, an overall reduction in the extracellular matrix (ECM) associated with a gradual decrease in collagen and elastin fiber production by fibroblasts and, on the other hand, by an increase in the destruction of these macromolecules by specific enzymes.

These biological phenomena therefore result in the induction, in the skin, of considerable physical modifications: loss of firmness, sagging. The skin loses its elasticity and the features slacken. The slackening of the subcutaneous tissues (fats and muscles) leads to an excess of skin and ptosis. This slackening is characterized by drooping of the cheekbones and of the cheeks, entraining the lower eyelid.

Also, the total amount of the extracellular matrix component collagen has been shown to be reduced with age. In contrast to those in young skin, which has abundant, tightly packed, and well-organized intact collagen fibrils, collagen fibrils in aged skin are fragmented and coarsely distributed. Increased collagen degradation and reduced collagen biosynthesis are both involved in this aberrant collagen homeostasis, which results in a net collagen deficiency. There is also increased collagen fragmentation, which is believed to be due to increased matrix metalloproteinase (MMP) expression in older skin. MMPs-2, -3, -9, -12, and -13 are capable of catabolizing elastic fibers. MMP-12, also known as human macrophage metalloelastase, is the most active protease in elastin degradation. Consequent elastin fragmentation results in reduced skin elasticity and the classical sign of skin aging, wrinkling Dermal fibroblasts contribute to age-associated dermal thinning as they are reduced in size, make less pro-collagen and have increased expression of MMP-1 with age, contributing to increased collagen fragmentation.

There is still a need in the state of the art to provide compositions that cosmetically target skin aging signs and that can be used in the treatment of diseases associated with skin.

### SUMMARY OF THE INVENTION

The object of the present invention is to provide a biologic skin care composition.

The authors of the present invention have developed a novel skin care composition comprising natural compounds that can be found in algae. In particular, they have developed a novel skin care composition comprising an extract of Haematococcus pluvialis and a polysaccharide.

Thus, in a first aspect the present invention relates to an extract of an Haematococcus pluvialis culture which is obtainable by a method that comprises the steps of:
(i) Preparing a lysate from an Haematococcus pluvialis culture,
(ii) Treating the lysate obtained in step (i) under acidic conditions in order to generate an acid soluble fraction and an acid insoluble pellet,
(iii) Separating the acid soluble fraction obtained in step (ii) from the acid insoluble pellet obtained in step (ii),
(iv) hydrolyzing the acid insoluble pellet obtained in step (iii),
(v) combining the acid soluble fraction obtained in step (iii) and the hydrolyzed pellet obtained in step (iv), thereby obtaining the extract and, optionally
(vi) neutralizing the extract obtained in step (v).

In a second aspect the present invention relates to a composition comprising the extract according to the first aspect and a polysaccharide or a glycosaminoglycan.

In a third aspect, the present invention relates to a cosmetic composition or cosmeceutical comprising the extract according to the first aspect or the composition according to the second aspect and a cosmetically acceptable vehicle.

In a fourth aspect, the present invention relates to a cosmetic method for skin care which comprises the topical administration to a subject of the cosmetic composition or cosmeceutical according to the third aspect.

In a fifth aspect, the present invention relates to a pharmaceutical composition comprising the extract according to the first aspect or the composition according to any of the second aspect and a pharmaceutically active adjuvant.

In a sixth aspect, the present invention relates to a method for preparing an extract of a Haematococcus pluvialis culture comprising the steps of the first aspect.

In a seventh aspect, the present invention relates to a method for increasing the collagen content of the skin in a subject comprising the topical administration of the extract according to the first aspect or the composition according to the second aspect.

In an eight aspect, the present invention relates to a method for increasing the elastin content of the skin in a subject comprising the topical administration to the subject of the extract according to the first aspect or the composition according to the second aspect.

### DESCRIPTION OF THE FIGURES

FIG. 1: Immunohistochemistry of Collagen I in in vitro skin tissue.

FIG. 2: Immunohistochemistry of Elastin in in vitro skin tissue.

FIG. 3: Summary table showing the water retention measured by transepithelial/transendothelial electrical resistance (TEER) in the presence of the product, hyaluronic acid and a composition comprising the product and hyaluronic acid.

FIG. 4: Graphical representation of the Skin Hydration with Corneometer after 10 minutes (T10′), 30 minutes (T30′), 1 hour (T1 h) and 24 hours (T24 h).

FIG. 5: Graphical representation of the Grey Index after 10 minutes (T10′), 30 minutes (T30′), 1 hour (T1 h) and 24 hours (T24 h).

FIG. 6: Graphical representation of the Anisotropy Index after 10 minutes (T10′), 30 minutes (T30′), 1 hour (T1 h) and 24 hours (T24 h).

FIG. 7: Graphical representation of the Gloss parameter after 10 minutes (T10′), 30 minutes (T30′), 1 hour (T1 h) and 24 hours (T24 h).

FIG. 8: Graphical representation of the Wrinkle depth after 10 minutes (T10′), 30 minutes (T30′), 1 hour (T1 h) and 24 hours (T24 h).

FIG. 9: Graphical representation of the Wrinkle volume after 10 minutes (T10′), 30 minutes (T30′), 1 hour (T1 h) and 24 hours (T24 h).

### DETAILED DESCRIPTION OF THE INVENTION

The authors of the present invention have found that extracts from Haematococcus pluvialis are suitable for promoting collagen and elastin production by the skin and thus, are suitable as cosmetic for promoting skin rejuvenation as well as for the treatment of certain skin diseases such as erythema.

Without wishing to be bound by any theory, it is thought that the cosmetic and therapeutic effects of the extract are the result of the presence in the extracts of certain polyamines, such as spermidine, as well as of certain amino acids which are those found in collagen.

Haematococcus pluvialis Extract

In a first aspect, the invention relates to an extract of a Haematococcus pluvialis culture which is obtainable by a method that comprises the steps of
(i) Preparing a lysate from an Haematococcus pluvialis culture,
(ii) Treating the lysate obtained in step (i) under acidic conditions in order to generate an acid soluble fraction and an acid insoluble pellet,
(iii) Separating the acid soluble fraction obtained in step (ii) from the acid insoluble pellet obtained in step (ii),
(iv) hydrolyzing the acid insoluble pellet obtained in step (iii),
(v) combining the acid soluble fraction obtained in step (iii) and the hydrolyzed pellet obtained in step (iv), thereby obtaining the extract and, optionally
(vi) neutralizing the extract obtained in step (v).

As used herein, Extract” is understood to mean generally a substance isolated vegetal, obtained by extraction from a plant raw material, preexisted and which does not in nature as such.

As used herein, the term “extract” may be any substance made by extracting a biomass of Haematococcus pluvialis. In accordance with the present disclosure, the extract is a solvent-based extract obtained by solvent extraction of the said algae. As used herein, extracting is achieved by using at least one solvent, preferably a polar solvent. Accordingly, in a preferred embodiment, the extract is obtained by performing a solvent extraction method. In a preferred embodiment, the extract is obtained from aqueous and/or hydroalcoholic extraction methods including cold or hot extraction, ultrasonic extraction, reflux cooling, needle extraction and microwaves extraction. However, the solvent used as well as the method are not limited thereto as long as an extract providing the inherent benefit effects in accordance with the spirit of the present disclosure may be obtained. The solvent may be added to the algal material. This may be achieved by any means known in the field such as, e.g., filtration, sieving, ultrafiltration, cross-flow filtration, centrifugation, precipitation over time, or a combination of two or more thereof.

As used herein, “Haematococcus pluvialis” is a freshwater species of Chlorophyta from the family Haematococcaceae.

The term “culture”, as used in the present invention, refers to a population of living Haematococcus pluvialis cells. Cultures for use in the present invention can be obtained by a person skilled in the art using conventional means, such as by farming, open ponds and photobioreactors. In some embodiments, the culture comprises both the Haematococcus pluvialis cells and the material secreted by the cells which is found in the culture medium. In other embodiments, the term culture is used to refer to the Haematococcus pluvialis biomass after their separation from the culture medium and from the secreted materials.

The term “biomass”, as used herein, includes biological material comprising, or deriving from, living or recently living organisms. By extension, the term includes not only the biological material or organic matter which constitutes an organism, but also the biological material or organic matter generated in a biological process, spontaneous or not spontaneous (i.e., provoked).

In a first step (step (i)), the process used for preparing the extract according to the invention comprising the preparation of a lysate from a Haematococcus pluvialis culture.

By “lysate” is understood in particular to mean a material obtained by cell lysis of the cells of the microorganism considered, thus causing the release of the intracellular biological constituents naturally contained in said cells. The lysate used is formed by all or part of the intracellular biological constituents and constituents of the cell walls and membranes. The present invention thus relates to the implementation of a lysate microorganism and/or a fraction thereof and/or one of its metabolites.

Methods for preparation of the lysate are, but not limited, cavitation, mechanical lysis, chemical lysis, enzymatic lysis, detergent lysis, osmotic lysis, ultrasonic lysis, freeze-thaw lysis, electric lysis, microfluidic lysis and laser lysis.

In a second step (step (ii)) of the process used for the preparation of the extract according to the invention, the lysate obtained in step (i) is treated under acidic conditions in order to generate an acid soluble fraction and an acid insoluble pellet.

The treatment of the lysate under acidic conditions comprise a treatment at pH values within a range of pH values of from about 1 to about 5. In some embodiments, the acidic conditions comprise the treatment of the lysate at a pH value of between about 0.1 and about to 6.9, between 0.2 to about 6.8, between about 0.3 to about 6.7, between about 0.4 and about 6.6, between about 0.5 and about 6.5, between about 0.6 to 6.4, between about 0.7 and about 6.3, between about 0.8 and about 6.2, between about 0.9 to about 6.1, between about 1 to about 6, between about 1 to about 5.9, between about 1 to about 5.8, 1 between about 1 and about 5.7, between about 1 and about 5.6, between about 1 and about 5.5, between about 1 and about 5.4, between about 1 and about 5.3, between about 1 and about 5.2, between about 1 and about 5.1, between about 1 and about 5, between about 1 and about 5.

The term “pellet” as used herein, refers to a sedimented portion that accumulates during algae centrifugation. In this particular case, the pellet is insoluble.

In a third step (step (iii)) of the process used for the preparation of the extract according to the invention, the acid soluble fraction obtained in step (ii) is separated from the acid insoluble pellet obtained in step (ii).

The separation of the soluble fraction and the insoluble can be carried out by any method known in the art, such as centrifugation, decanting and filtration.

In a fourth step (step (iv)) of the process used for the preparation of the extract according to the invention, the acid insoluble pellet obtained in step (iii) is hydrolyzed.

As used herein, the term “hydrolysis” means any chemical reaction in which a molecule of water breaks one or more chemical bonds. The term is used broadly for substitution, elimination, and solvation reactions in which water is the nucleophile. Suitable methods for the hydrolysis of the pellet, but not limited, comprise thermal hydrolysis, enzymatic hydrolysis, alkaline hydrolysis or acid hydrolysis.

In a fifth step (step (v)) of the process used for the preparation of the extract according to the invention, the acid soluble fraction obtained in step (iii) and the hydrolyzed pellet obtained in step (iv) are combined, thereby obtaining the extract

In a sixth step (step (vi)) of the process used for the preparation of the extract according to the invention, the extract obtained in step (v) is optionally neutralized.

Herein the term “neutralization” is understood as the adjustment of the pH to approach to the pH optimum of the composition.

In a particular embodiment, the extract comprises collagen amino acids

The term “collagen amino acids”, as used herein, refers to amino acids which predominantly form collagen. The term includes proline, glycine, hydroxyproline and any combination thereof.

In some embodiments, the extract according to the present invention comprises from about 0.50% to about 50.00%, from about 1.00% to about 49.00%, from about 1.50% to about 48.00%, from about 2.00% to about 47.00%, from about 2.50% to about 46.00%, from about 3.00% to about 45.00%, from about 3.50% to about 44.00%, from about 4.00% to about 43.00%, from about 4.50% to about 42.00%, from about 5.00% to about 41.00%, from about 5.50% to about 40.00%, from about 6.00% to about 39.00%, from about 6.50% to about 38.00%, from about 7.00% to about 37.00%, from about 7.50% to about 36.00%, from about 8.00% to about 35.00%, from about 8.50% to about 34.00%, from about 9.00% to about 33.00%, from about 9.50% to about 32.00%, from about 10.00% to about 31.00%, from about 10.00% to about 30.00% of proline, all the values provided in % by weight.

In some embodiments, the extract according to the present invention comprises from about 0.50% to about 50.00%, from about 1.00% to about 49.00%, from about 1.50% to about 48.00%, from about 2.00% to about 47.00%, from about 2.50% to about 46.00%, from about 3.00% to about 45.00%, from about 3.50% to about 44.00%, from about 4.00% to about 43.00%, from about 4.50% to about 42.00%, from about 5.00% to about 41.00%, from about 5.50% to about 40.00%, from about 6.00% to about 39.00%, from about 6.50% to about 38.00%, from about 7.00% to about 37.00%, from about 7.50% to about 36.00%, from about 8.00% to about 35.00%, from about 8.50% to about 34.00%, from about 9.00% to about 33.00%, from about 9.50% to about 32.00%, from about 10.00% to about 31.00%, from about 10.00% to about 30.00% of glycine, all the values provided in % by weight.

In some embodiments, the extract according to the present invention comprises from about 0.50% to about 50.00%, from about 1.00% to about 49.00%, from about 1.50% to about 48.00%, from about 2.00% to about 47.00%, from about 2.50% to about 46.00%, from about 3.00% to about 45.00%, from about 3.50% to about 44.00%, from about 4.00% to about 43.00%, from about 4.50% to about 42.00%, from about 5.00% to about 41.00%, from about 5.50% to about 40.00%, from about 6.00% to about 39.00%, from about 6.50% to about 38.00%, from about 7.00% to about 37.00%, from about 7.50% to about 36.00%, from about 8.00% to about 35.00%, from about 8.50% to about 34.00%, from about 9.00% to about 33.00%, from about 9.50% to about 32.00%, from about 10.00% to about 31.00%, from about 10.00% to about 30.00% of hydroxyproline, all the values provided in % by weight.

In some embodiments, the extract according to the present invention, the combined content of the collagen amino acids (proline, glycine and hydroxyproline) in the composition of the invention is from about 0.50% to about 50.00%, from about 1.00% to about 49.00%, from about 1.50% to about 48.00%, from about 2.00% to about 47.00%, from about 2.50% to about 46.00%, from about 3.00% to about 45.00%, from about 3.50% to about 44.00%, from about 4.00% to about 43.00%, from about 4.50% to about 42.00%, from about 5.00% to about 41.00%, from about 5.50% to about 40.00%, from about 6.00% to about 39.00%, from about 6.50% to about 38.00%, from about 7.00% to about 37.00%, from about 7.50% to about 36.00%, from about 8.00% to about 35.00%, from about 8.50% to about 34.00%, from about 9.00% to about 33.00%, from about 9.50% to about 32.00%, from about 10.00% to about 31.00%, from about 10.00% to about 30.00% all the values provided in % by weight.

In a particular embodiment, the extract comprises spermidine.

“Spermidine”, as used herein, (CAS Number-124-20-9) is a biogenic polyamine from which spermine can be derived.

In a preferred embodiment, the extract comprises between about 0,001% and about 1% (w/w) of spermidine. More preferably, the extract comprises about 0% to about 100%, about 0.0001% to about 95%, about 0.0002% to about 90%, about 0.0003% to about 85%, about 0.0004% to about 80%, about 0.0005% to about 75%, about 0.0006% to about 70%, about 0.0007% to about 65%, about 0.0008% to about 60%, about 0.0009% to about 55%, about 0.00091% to about 50%, about 0.00092% to about 45%, about 0.00093% to about 40%, about 0.00094% to about 35%, about 0.00095% to about 30%, about 0.00096% to about 25%, about 0.00097% to about 20%, about 0.00098% to about 15%, about 0.00099% to about 10%, about 0.000995% to about 5%, about 0.001% to about 1%, all the values given in % by weight.

Compositions Comprising an Haematococcus pluvialis Extract and a Polysaccharide/Glycosaminoglycan

In a second aspect, the present invention relates to a composition comprising the extract according to the first aspect and a polysaccharide or a glycosaminoglycan.

“Combination” or “composition”, as used herein, indicates that the Haematococcus pluvialis microalgae extract containing polyamines such as spermidine and collagen amino acids and polysaccharide/glycosaminoglycan. It will be understood that the two components of the formulation can be provided as single formulation wherein both components are found in a single composition or, alternatively, can be provided as a kit-of-parts wherein each of the components are separately formulated and are brought in combination at the moment of application.

The term “polysaccharide”, as used herein, refers to a long chain polymeric carbohydrate composed of monosaccharide units bound together by glycosidic linkages. Suitable polysaccharides that can be found in the combinations according to the present invention include, without limitation, glycosaminoglycan, starch, cellulose, galacturonic acid-rich polysaccharides and glucosamine-rich polysaccharides.

As used herein, “Glycosaminoglycans” or “GAGs” or “mucopolysaccharides” are negatively-charged polysaccharide compounds. They are composed of repeating disaccharide units that are present in every mammalian tissue. Suitable GAGs than can form part of the compositions according to the present invention include, without limitation, heparin/heparan sulfate, chondroitin sulfate/dermatan sulfate, keratan sulfate, and hyaluronic acid.

In a preferred embodiment, the glycosaminoglycan used in this invention is hyaluronic acid.

The term “hyaluronic acid” (HA), also known as hyaluronan or hyaluronate, (CAS Number 9004-61-9) refers to a high molecular weight polysaccharide (glycosaminoglycan).

HA is composed of repeating disaccharide units of glucuronic acid (GlcA) and N-acetylglucosamine (GlcNAc), as depicted below:

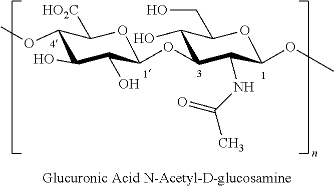

The composition according to the invention may contain any form of hyaluronic acid that is suitable for the elastin-boosting and collagen-boosting effects according to the invention. The composition according to the present invention may a single type of hyaluronic acid or a mixture of different types of hyaluronic acid, wherein each type of hyaluronic acid is defined by a certain range of number average molar weights (Mn) or mass average molar weights (Mw).

In some embodiments, the composition comprises a first type of hyaluronic acid, said first type being characterized by a Mn or Mw which is from 100 kDa to 300 kDa, from 110 kDa to 290 kDa, from 120 kDa to 280 kDa, from 130 kDa to 270 kDa, from 140 kDa to 260 kDa, from 150 kDa to 250 kDa, from 160 kDa to 240 kDa, from 170 kDa to 230 kDa, from 180 kDa to 220 kDa, from 190 kDa to 210 kDa or of about 200 kDa.

In some embodiments, the composition comprises a second type of hyaluronic acid, said second type being characterized by a Mn or Mw which is from 20 kDa to 50 kDa, from 22 kDa to 48 kDa, from 24 kDa to 46 kDa, from 26 kDa to 44 kDa, from 28 kDa to 42 kDa, from 30 kDa to 40 kDa, from 32 kDa to 38 kDa, from 34 kDa to 36 kDa or of about 35 kDa.

In some embodiments, the composition comprises a third type of hyaluronic acid, said third type being characterized by a Mn or Mw which is from 1 kDa to 20 kDa, from 2 kDa to 18 kDa, from 4 kDa to 16 kDa, from 6 kDa to 14 kDa, from 8 kDa to 12 kDa, from 10 kDa to 11 kDa or of about 11 kDa.

In some embodiments, the composition comprises a mixture of the first and second type of hyaluronic acid as defined above.

In some embodiments, the composition comprises a mixture of the first and third type of hyaluronic acid as defined above.

In some embodiments, the composition comprises a mixture of the second and third type of hyaluronic acid as defined above.

In some embodiments, the composition comprises a mixture of the first, second and third type of hyaluronic acid as defined above.

### Cosmetic and Cosmeceutical Compostions and Uses Thereof

In a third aspect, the present invention relates to a cosmetic composition or cosmeceutical comprising the extract according to the first aspect or the composition according to the second aspect and a cosmetically acceptable vehicle.

As used herein, the term “cosmetic composition” and “skin care composition” are used herein interchangeably to refers to a composition suitable for use in order to enhance the natural beauty or change the body appearance without affecting the structure or functions of the human or animal body, comprising one or more products providing such effects. If desired, the cosmetic composition provided by the invention can contain, in addition to the algal extract of the invention, one or more cosmetic products, i.e., substances or mixtures intended to be placed in contact with the skin for the exclusive or main purpose of the skin care is skin rejuvenation, protection of the skin from dehydration, reduction of the effects associated with aging or reducing wrinkles. Illustrative examples of cosmetically acceptable vehicles include the products contained in the INCI (International Nomenclature of Cosmetic Ingredients) list. The extract or composition according to the present invention may be added to a wide variety of products for cosmetic application, including makeup, creams for cleansing, protecting, treating, or caring for the skin, in particular, the face, hands, and feet (e.g., day and night creams, makeup removal creams, foundation creams and sunscreens), liquid foundations, makeup removal lotions, protective or skin-care body lotions, sunscreen lotions, skin care lotions, gels, or foams, such as cleansing, sunscreen, and artificial tanning lotions, bath preparations, deodorant compositions, after-shave gels or lotions, depilatory creams, and compositions used for insect stings and against pain. The combination of the invention may take any of a wide variety of forms, and include, for example dressings, lotions, solutions, sprays, creams, gels, ointments, or the like.

As used herein, the term “cosmeceutical product” refers to a product suitable for use in the body or animal body comprising one or more cosmeceutical products (functional cosmetics, dermaceuticals or active cosmetics), i.e., topical hybrid products with cosmetic-pharmaceutical characteristics containing active ingredients having effect on user's skin at higher and more effective concentrations, therefore they are located in an intermediate level between cosmetic and drug. Illustrative examples of cosmeceutical products include essential oils, ceramides, enzymes, minerals, amino acids, vitamins, etc.

The term “cosmetically acceptable vehicle or carrier” as used herein refers to a substantially non-toxic carrier, useable for administration of cosmetics, with which active compounds will remain stable and bioavailable. The carrier must be of sufficiently high purity and of sufficiently low toxicity to render it suitable for administration to the mammal being treated. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components. It further should maintain the stability and bio availability of an active agent. The cosmetically acceptable carrier is selected with the planned manner of administration in mind, to provide for the desired bulk, consistency, etc., when combined with an active agent and other components of a given composition.

The term “cosmetic” as used herein refers to articles (excluding soap) intended to be rubbed, poured, sprinkled, or sprayed on, introduced into, or otherwise applied to the human body or any part thereof for cleansing, beautifying, promoting attractiveness, or altering the appearance, and articles intended for use as a component of any such articles.

The cosmetic and cosmeceutical compositions according to the invention are then used for skin care by contacting or applying an effective amount of the composition with the part of the skin wherein the cosmetic effect is desired.

The term “contact” and its various grammatical forms as used herein refers to a state or condition of touching or of immediate or local proximity.

The term “cosmetic effect” as used herein refers to a consequence of applying a cosmetic to the skin with the intention of improving is appearance or of beautifying it.

The term “apply” as used herein refers to placing in contact with or to lay or spread on. The term “administer” as used herein means to give or to apply. The term “administering” as used herein includes in vivo administration, as well as administration directly to tissue ex vivo.

The term “subject”, as used herein, relates to an animal, preferably a vertebrate, more preferably a mammal, preferably to a livestock, like a cattle, a horse, a pig, a sheep, or a goat, to a companion animal, such as a cat or a dog, or to a laboratory animal, like a rat, mouse, or guinea pig. Most preferably, the subject is a human.

The term “effective amount” of cosmetic agent refers to a substantially nontoxic but sufficient amount of a compound to provide the desired effect and performance at a reasonable benefit/risk ratio attending any medical or cosmetic treatment.

By “topical application” is understood to mean, within the meaning of the present invention an application to the skin (including the scalp), mucous membranes and/or the hair.

By “epidermal barrier” is understood to mean, within the meaning of the present invention, the cellular structures of the epidermis, in particular the barrier formed by the corneocytes tissue and cement intercellular lipid.

By “barrier function of the skin barrier function” or “epidermal” is understood, within the meaning of the present invention, the protective function of the epidermis, in particular against outer attacks, and controlling the transepidermal water loss and ions.

In a preferred embodiment, the skin care is skin rejuvenation, protection of the skin from dehydration, reduction of the effects associated with aging or reducing wrinkles.

“Signs of skin aging” include, but are not limited to, all outwardly visible or tactilely perceptible manifestations as well as any other macro or micro effects due to skin aging. Such signs may be induced or caused by intrinsic factors or extrinsic factors, e.g., chronological aging and/or environmental damage. These signs may result from processes that include, but are not limited to, the development of textural discontinuities such as wrinkles and coarse deep wrinkles, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including under eye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

“Skin rejuvenation” means a process of reducing, diminishing, retarding or reversing one or more signs of skin aging or generally improving the condition of skin. For instance, skin rejuvenation may include increasing luminosity of the skin, reducing pore size, reducing fine lines or wrinkles, improving thin and transparent skin, improving firmness, improving sagging skin (such as that produced by bone loss), improving dry skin (which might itch), reducing or reversing freckles, age spots, spider veins, reducing or preventing the appearance of rough and leathery skin, fine wrinkles that disappear when stretched, reducing loose skin, or improving a blotchy complexion. According to the present disclosure, one or more of the above conditions may be improved or one or more signs of aging may be reduced, diminished, retarded or even reversed by certain embodiments of the compositions, methods and uses of the present disclosure.

According to some of any of the embodiments described herein the “dehydration” is effected by freeze drying, heat drying, vacuum drying or any combination thereof.

As used herein, “reducing wrinkles” means preventing, retarding, arresting, or reversing the process of wrinkle formation in human skin.

The term “wrinkle”, as used herein, refers to a sign of aging of the skin associated with a loss of elasticity or structural integrity of the skin tissue, including but not limited to sagging, lax and loose tissue, and resulting in the presence of lines, including fine lines, fine wrinkles, or coarse wrinkles in the skin. Examples of wrinkles include, but are not limited to, lines around the eyes (e.g. “crow's feet”), brow droop, tear troughs, bunny lines, forehead and check, frown lines, nasolabial folds, vertical lip lines.

### Pharmaceutical Compositions

In a fifth aspect, the invention relates to a pharmaceutical product comprising the extract according to the first aspect or the composition according to the second aspect and a pharmaceutically active adjuvant.

The term “pharmaceutical composition”, as used herein, relates to compositions and molecular entities that are physiologically tolerable. The pharmaceutical compositions forms are not limited to creams, ointments, gels, paste, powder, sponge, tape, tincture, solutions, suspensions, injectables, patches, tablets or capsules. Preferably, the term “pharmaceutically acceptable” means it is approved by a regulatory agency of a state or federal government or is included in the U.S. Pharmacopeia or other generally recognized pharmacopeia for use in animals, and more particularly in humans.

A non-exclusive list of pharmaceutical excipients (polymers, surfactants, dispersants, solubilizers, bulking agents, etc.) includes, but is not limited to, polyvinylpyrrolidone, polyvinyl acetate, polysorbate, polyethylene glycol, polyvinyl alcohol, polyvinyl alcohol-polyethylene glycol, Poloxamer (polyethylene glycol-block-polypropylene glycol-block-polyethylene glycol), hydrogenate castor oils, and Mygliols. An extensive, but not exhaustive, list of pharmaceutical excipients can be found in the Handbook of Pharmaceutical Excipients. edited by Ainley Wade and Paul J.

As used herein, the terms “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The terms “compositions,” “pharmaceutical compositions,” “formulations,” “pharmaceutical formulations,” “preparations”, and “pharmaceutical preparations” are used synonymously and interchangeably herein.

### Medical Uses

In a fifth aspect of the invention, all previous extracts and compositions have a use in medicine.

In a particular embodiment, the medical use is in the treatment of erythema.

The term “treatment”, as used herein, refers to any type of therapy, which is aimed at terminating, preventing, ameliorating or reducing the susceptibility to a clinical condition as described herein. In a preferred embodiment, the term treatment relates to prophylactic treatment (i.e. a therapy to reduce the susceptibility to a clinical condition), of a disorder or a condition as defined herein. Thus, “treatment,” “treating,” and their equivalent terms refer to obtaining a desired pharmacologic or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse effect attributable to the disorder.

The term “therapeutically effective amount”, as used herein, relates to the sufficient amount of a compound (i.e. of the combination of the invention) to provide the desired effect and it will generally be determined, by among other causes, the characteristics of the compound itself and the therapeutic effect to be achieved. It will also depend on the subject to be treated, the severity of the disease suffered by said subject, the chosen dosage form, administration route, etc. For this reason, the person skilled in the art must adjust the doses depending on the aforementioned variables.

As used herein, the term “erythema” or “skin erythema” means skin redness, normally caused by skin sensitivity, occurring in healthy individuals and not linked to pathological disorders.

Method for Preparing the Haematococcus pluvialis Extract

In a sixth aspect, the invention relates to a method for preparing an extract of an Haematococcus pluvialis culture comprising the steps of
(i) Preparing a lysate from an Haematococcus pluvialis culture,
(ii) Treating the lysate obtained in step (i) under acidic conditions in order to generate an acid soluble fraction and an acid insoluble pellet,
(iii) Separating the acid soluble fraction obtained in step (ii) from the acid soluble pellet obtained in step (ii),
(iv) hydrolyzing the acid insoluble pellet obtained in step (ii) and
(v) combining the soluble fraction obtained in step (iii) and the hydrolyzed pellet obtained in step (iv), thereby obtaining the extract and, optionally
(vi) neutralizing the extract obtained in step (v).

All the different embodiments of the steps (i) to (vi) of the method for preparing the Haematococcus pluvialis extract have been defined above in the context of the Haematococcus pluvialis extracts according to the invention and are equally applicable to the method

In a preferred embodiment, the acidic conditions used in step (ii) comprise a treatment at pH values of between 1 and 5.

More preferably, the hydrolysis which is carried out in step (iv) is a thermal hydrolysis, an enzymatic hydrolysis or a chemical hydrolysis.

In a particular embodiment, the method further comprises admixing the extract obtained in step (vi) with a polysaccharide or with a glycosaminoglycan.

In a preferred embodiment, the concentration of polysaccharide or glycosaminoglycan in the composition is of between about 5% to 95% p/p of the composition.

More preferably, the glycosaminoglycan is hyaluronic acid.

### Method for Increasing the Skin Collagen Content

In a seventh aspect, the invention relates to a method for increasing the collagen content of the skin in a subject comprising the topical administration of the extract according to the first aspect or the composition according to the second aspect.

The term “collagen”, as used herein, (CAS Number 9064-67-9) is a main protein component constituting connective tissue in animals and is characterized by having a collagen triple helical structure. In some embodiments, the collagen boosting effect A total of not less than 30 types of collagens have been reported which are respectively termed Type I, Type II, and so on. Type I collagen is the primary component of the derma, ligaments, tendons, bones and the like; and Type II collagen is the primary component of articular cartilage. Type Ill collagen is found in the skin, lungs, intestinal walls, and the walls of blood vessels. Further, Type IV collagen is mainly contained in a basal membrane, which is the undercoat of all epithelial tissues. Type I collagen is the most abundant collagen in the body. The term collagen includes the various types of collagens.

Suitable assays for determining the collagen content and whether the composition is actually producing the collagen-boosting effect are well known to the person skilled in the art and, include. among others, measurement by immunohistochemistry and quantification of the signal as described in Example 1.

### Method for Increasing the Skin Elastin Content

In an eight aspect, the invention relates to a method for increasing the elastin content of the skin in a subject comprising the topical administration of the extract according to the first aspect or the composition according to the second aspect.

The term “elastin”, as used herein, refers to any type of elastin that is produced and deposited in any form in skin of a subject. In particular, the term “elastin” refers to mammalian, preferably human, elastin.

Suitable assays for determining the elastin content and whether the extract or composition is actually producing the elastin-boosting effect are well known to the person skilled in the art and, include. among others, measurement by immunohistochemistry and quantification of the signal as described in Example 1.

In some embodiments, the method for increasing collagen content or elastin content according to the invention are in vitro methods. In some embodiments, the method for increasing collagen content or elastin content is carried out by topical application of the extract or composition to a subject wherein said collagen-boosting effect is desired.

In some embodiments of the methods for increasing collagen or elastin according to the present invention, the extract or composition according to the present invention may be applied to any skin areas which have developed lines and/or wrinkles, or any skin areas which are susceptible to the adverse effects of the environment, daily stress, sun exposure, or premature aging, and which may be expected to develop lines and/or wrinkles. The products are particularly suited to address those skin areas which are most resistant to skin remodeling, such as the crow's feet in the skin of the periorbital regions around the eyes.

In some embodiments of the methods for increasing collagen or elastin according to the present invention, the extract or composition according to the present invention may be applied to the skin on an as-needed basis or according to a pre-set schedule. The composition may be applied directly to clean skin, before application of any other treatment product, or foundation makeup, or they may be applied over the other treatment product or foundation makeup. The amount of the

In some embodiments of the methods for increasing collagen or elastin according to the present invention, the application of the extract or composition according to the present invention to the skin with each application can vary widely depending on the specific need of the user. For example, if the user has prominent wrinkles, the user may choose to apply the compositions more frequently than if the user's skin exhibits finer lines. The composition may be applied for a period of days to months or even years, and at a frequency ranging from about once or twice a day to once a week. In another example, the composition may be applied one or twice a day, morning and/or evening, for a period of six months or more.

The invention is described below by means of the following examples which are to be construed as merely illustrative and not limitative of the scope of the invention.

### EXAMPLES

### Materials

### Haematococcus Extract

Haematococcus pluvialis biomass was subjected to cell lysis. The suspension was separated by centrifugation and the solid residue was discarded. The protein fraction from the supernatant was precipitated under acidic conditions and the resulting suspension was centrifuged. After centrifugation, the supernatant was neutralized to obtain the first microalgae extract. The protein pellet was hydrolyzed, neutralized, and combined with the first extract to obtain the desired final extract.

### Determination of Polyamine Composition by HPLC Analysis

The polyamine composition and quantification were performed by HPLC following the method described by Morgan (“Determination of polyamines as their benzoylated derivatives by HPLC”, Methods Mol Biol, 1998, 79, 111-118). In this method, the sample was mixed with an internal standard and the mixture reacts with benzoyl chloride. The corresponding benzyl derivative is analyzed by HPLC.

### Determination of Amino Acid Composition by HPLC Analysis

The amino acid composition and quantification were performed by HPLC adapting the method from Agilent Application Notes: “Improved Amino Acid Methods using Agilent ZORBAX Eclipse Plus C18 Columns for a Variety of Agilent LC Instrumentation and Separation Goals”. In this method the hydrolyzed protein mixture reacts with Fmoc-chloride and OPA to obtain their corresponding derivatives, which are analyzed by HPLC.

### Results

### Example 1—In Vitro Collagen and Elastin Boosting Effect

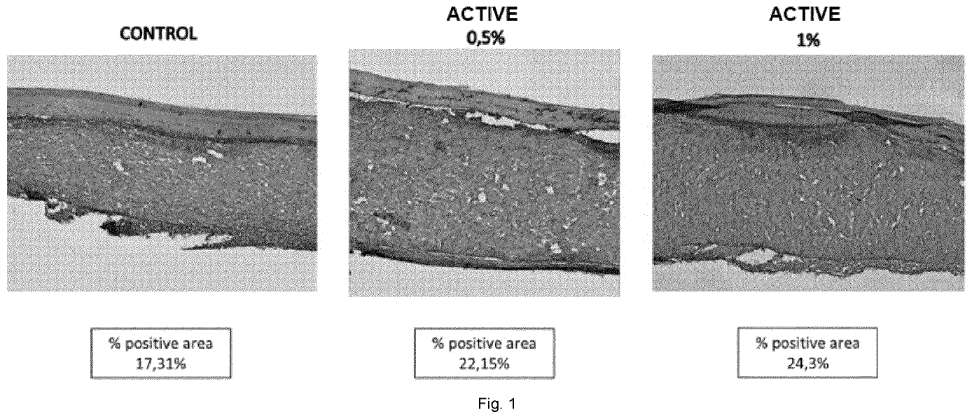

Collagen content in response to the treatment with the extract according to the invention (ACTIVE) was analysed by immunohistochemistry. FIG. 1 shows immunohistochemistry staining of Collagen I in in vitro skin tissue. Images are codified as CONTROL (no active ingredient present), ACTIVE 0.5% (active at a 0.5% concentration) and ACTIVE 1% (active at a 1% concentration). The “% positive area” shown below indicates immunohistochemistry positive reaction for Collagen I after 24-hour full-thickness skin treatment. CONTROL image has a positive reaction area of 17.31% (0% increase). ACTIVE 0.5% image has a positive reaction area of 22.15% (27.96% increase vs. control). ACTIVE 1% image has a positive reaction area of 24.3% (40.38% increase vs. control).

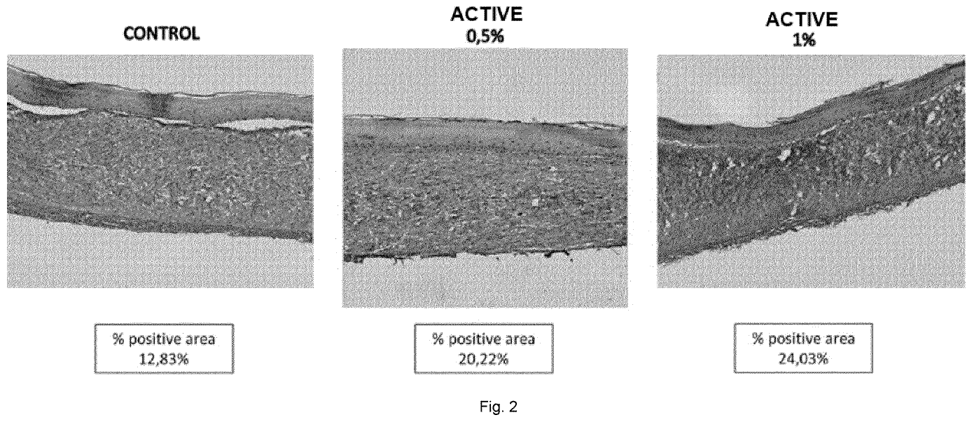

Elastin content in response to the treatment with the extract according to the invention (ACTIVE) was analysed by immunohistochemistry FIG. 2 shows Immunohistochemistry of Elastin in in vitro skin tissue. Images are codified as CONTROL (no active ingredient present), ACTIVE 0.5% (active at a 0.5% concentration) and ACTIVE 1% (active at a 1% concentration). The “% positive area” shown below indicates immunohistochemistry positive reaction for Elastin after 24-hour full-thickness skin treatment. CONTROL image has a positive reaction area of 12.83% (0% increase). ACTIVE 0.5% image has a positive reaction area of 20.22% (57.60% increase vs control). ACTIVE 1% image has a positive reaction area of 24.03% (89.60% increase vs control).

The results are summarized in the following Table

| Immunohisto- | % collagen | % elastin | % collagen | % elastin |
|---|---|---|---|---|
| chemistry | positive area | positive area | increase | increase |
| Control | 17.31 | 12.83 | 0 | 0 |
| Active 0.5% | 22.15 | 20.22 | 27.96 | 57.60 |
| Active 1% | 24.3 | 24.3 | 40.38 | 89.40 |

### Example 2—Dehydration Protection Effect. In Vitro TEER % Dehydration Protection

Results are reported as percentage of variation between the pre-treatment and post-dehydration versus the positive control (CTR+) (* p-value<0.05, Student T-test). The results are the following: active 0.5% has a 10.4% variation (34.34% water retention improvement), active 1% has a 12.9% variation (34.34% water retention improvement), hyaluronic acid 0.5% has a 11.1% variation (33.48% water retention improvement) and active 0.5%+hyaluronic acid 0.5% has a 19.9% variation (59.53% water retention improvement).

### Example 3—In Vivo Short-Term Effects at 3%

### 3.1 Skin Hydration (Corneometer)

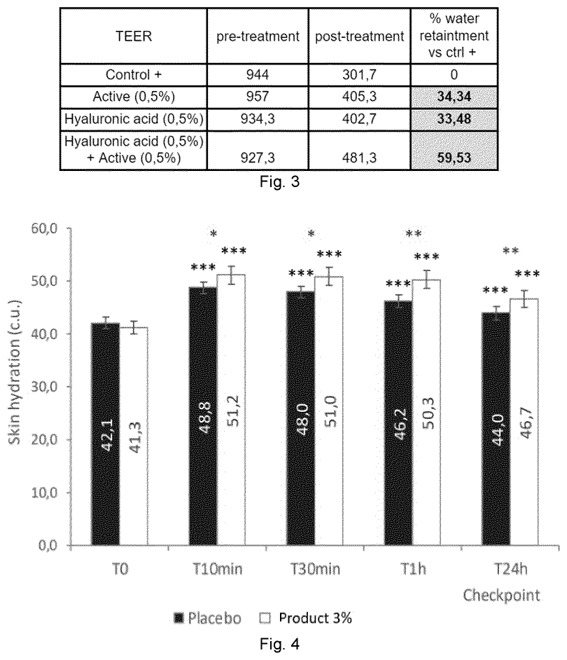

FIG. 4 shows the graphical representation of the Skin Hydration with Corneometer after 10 minutes (T10′), 30 minutes (T30′), 1 hour (T1 h) and 24 hours (T24 h). Statistical difference between active and placebo is shown (* p<0.05; **p<0.01;***p<0.001, Student's T test).

### 3.2 Grey Index (MoistureMap)

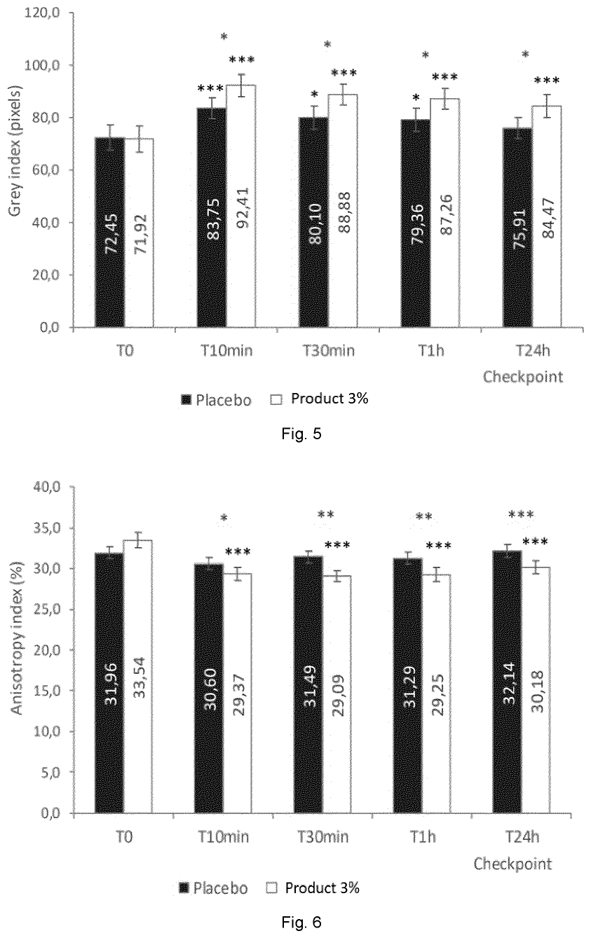

Moisture increases conductivity producing darker pixels (higher Gray Index values), while dryer skin is less conductive producing lighter pixels (lower Gray Index values). FIG. 5 shows the graphical representation of the Grey Index after 10 minutes (T10′), 30 minutes (T30′), 1 hour (T1 h) and 24 hours (T24 h). Statistical difference between active and placebo is shown (* p<0.05; **p<0.01;***p<0.001, Student's T test).

### 3.3 Anisotropy Index (MoistureMap)

The Anisotropy Index is an aging parameter based on the micro-topography of skin. The Anisotropy Index measures the directionality of the lines. The level of anisotropy increases with age (younger skin should have a lower anisotropy index than older skin). FIG. 6 shows the graphical representation of the Anisotropy Index after 10 minutes (T10′), 30 minutes (T30′), 1 hour (T1 h) and 24 hours (T24 h). Results are reported as relative difference from TO. Statistical difference between active and placebo is shown (* p<0.05; **p<0.01;***p<0.001, Student's T test).

### 3.4 Gloss Parameter

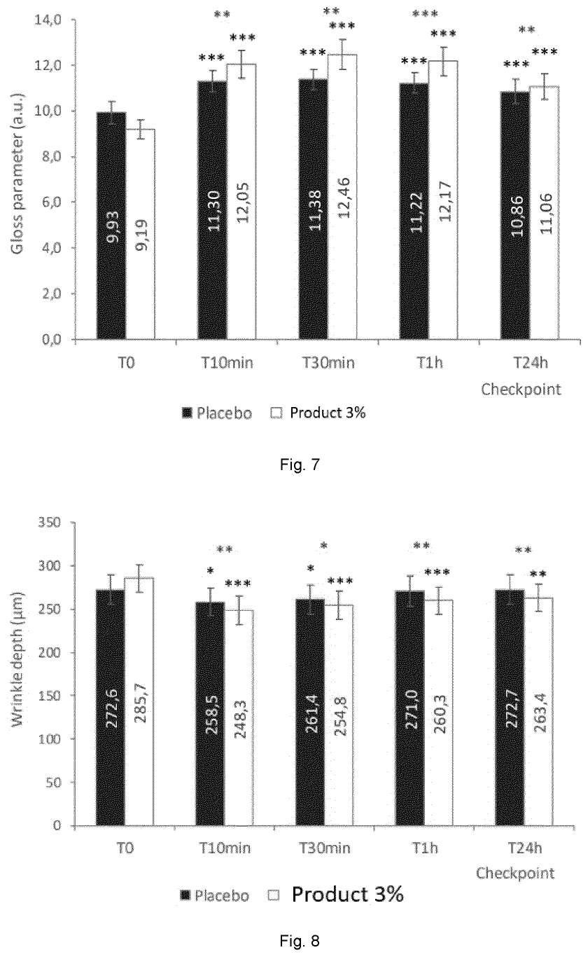

Skin radiance (or skin brightness) is the ability of the skin to reflect the light and it is measured using the gloss parameter (taken using the spectrophotometer/colorimeter CM-700D (Konica-Minolta)). FIG. 7 shows the graphical representation of the Gloss parameter after 10 minutes (T10′), 30 minutes (T30′), 1 hour (T1 h) and 24 hours (T24 h). Results are reported as relative difference from TO. Statistical difference between active and placebo is shown (* p<0.05; **p<0.01;***p<0.001, Student's T test).

### 3.5 Wrinkle Depth (3D Profilometry)

FIG. 8 shows the graphical representation of the Wrinkle depth after 10 minutes (T10′), 30 minutes (T30′), 1 hour (T1 h) and 24 hours (T24 h). Results are reported as relative difference from T0. Statistical difference between active and placebo is shown (* p<0.05; **p<0.01;***p<0.001, Student's T test).

### 3.6 Wrinkle Volume (3D Profilometry)

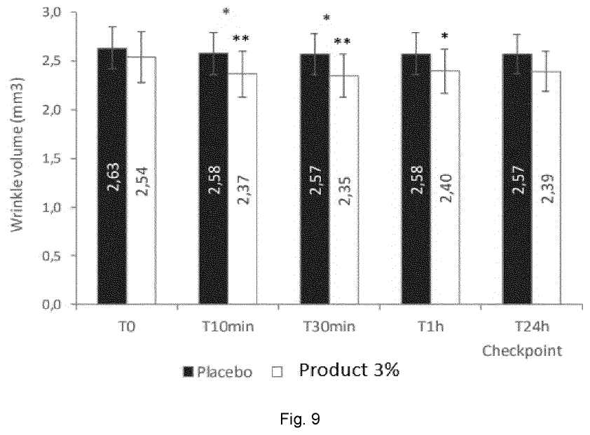

FIG. 9 shows the graphical representation of the Wrinkle volume after 10 minutes (T10′), 30 minutes (T30′), 1 hour (T1 h) and 24 hours (T24 h). Results are reported as relative difference from T0. Statistical difference between active and placebo is shown (* P<0.05; **p<0.01;***p<0.001, Students T test).

1. An extract of an Haematococcus pluvialis culture which is obtained by a method that comprises the steps of:
(i) preparing a lysate from an Haematococcus pluvialis culture,
(ii) treating the lysate obtained in step (i) under acidic conditions in order to generate an acid soluble fraction and an acid insoluble pellet,
(iii) separating the acid soluble fraction obtained in step (ii) from the acid insoluble pellet obtained in step (ii),
(iv) hydrolyzing the acid insoluble pellet obtained in step (iii),
(v) combining the acid soluble fraction obtained in step (iii) and the hydrolyzed pellet obtained in step (iv), thereby obtaining the extract and, optionally
(vi) neutralizing the extract obtained in step (v).

2. The extract according to claim 1, wherein the acidic conditions used in step (ii) comprise a treatment at pH values of between about 1 and about 5.

3. The extract according to claim 1, wherein the hydrolyzing in step (iv) is carried is a thermal hydrolysis, an enzymatic hydrolysis or a chemical hydrolysis.

4. The extract according to claim 1, wherein the extract comprises between about 1% to about 30% (w/w) of one or more amino acids selected from the group consisting of proline, hydroxyproline, glycine and alanine.

5. The extract according to claim 1, wherein the extract comprises between about 0,001% and about 1% (w/w) of spermidine.

6. A composition comprising the extract according to claim 1, and a polysaccharide or a glycosaminoglycan.

7. The composition according to claim 6, wherein the glycosaminoglycan is hyaluronic acid.

8. The composition according to claim 6, wherein the concentration of polysaccharide or glycosaminoglycan in the composition is of about 5% to 95% by weight of the composition.

9. A cosmetic composition or cosmeceutical comprising the extract according to claim 1 and a cosmetically acceptable vehicle.

10. A cosmetic method for skin care comprising administering topically the cosmetic composition or cosmeceutical according to claim 9 to a subject in need thereof.

11. The cosmetic method according to claim 10 wherein the skin care is skin rejuvenation, protection of the skin from dehydration, reduction of the effects associated with aging or reducing wrinkles.

12. A pharmaceutical composition comprising the extract according to claim 1 and a pharmaceutically active adjuvant.

13. (canceled)

14. A method of treating erythema comprising administering a therapeutically effective amount of the extract according to claim 1.

15. A method for preparing an extract of an Haematococcus pluvialis culture comprising the steps of:
(i) preparing a lysate from an Haematococcus pluvialis culture,
(ii) treating the lysate obtained in step (i) under acidic conditions in order to generate an acid soluble fraction and an acid insoluble pellet,
(iii) separating the acid soluble fraction obtained in step (ii) from the acid soluble pellet obtained in step (ii),
(iv) hydrolyzing the acid insoluble pellet obtained in step (ii) and
(v) combining the soluble fraction obtained in step (iii) and the hydrolyzed pellet obtained in step (iv), thereby obtaining the extract and, optionally
(vi) neutralizing the extract obtained in step (v).

16. The method according to claim 15, wherein the acidic conditions used in step (ii) comprise a treatment at pH values of between 1 and 5.

17. The method according to claim 15, wherein the hydrolyzing in step (iv) is carried is a thermal hydrolysis, an enzymatic hydrolysis or a chemical hydrolysis.

18. The method according to claim 15, further comprising admixing the extract obtained in step (v) with a polysaccharide or with a glycosaminoglycan.

19. The method according to claim 18, wherein the wherein the concentration of polysaccharide or glycosaminoglycan in the composition is of between about 5% to 95% p/p of the composition.

20. The method according to claim 19, wherein the glycosaminoglycan is hyaluronic acid.

21. A method for increasing the collagen content of the skin in a subject comprising topically administering the extract according to claim 1.

22. A method for increasing the elastin content of the skin in a subject comprising topically administering to subject in need thereof the extract according to claim 1.
